(19) United States
(12) Patent Application Publication
(10) Pub. No.: US 2015/0203807 A1
(43) Pub. Date: Jul. 23, 2015

(54) INACTIVATION OF A PRODUCTION STRAIN USING A FATTY ACID

(71) Applicant: NOVOZYMES A/S, Bagsvaerd (DK)

(72) Inventors: Helene Munthe Jensen, Bagsvaerd (DK); Martin Ellegaard, Bagsvaerd (DK); Sune Jakobsen, Bagsvaerd (DK)

(21) Appl. No.: 14/412,931

(22) PCT Filed: Jul. 2, 2013
(86) PCT No.: PCT/EP2013/063928

§ 371 Date: Jan. 5, 2015

Related U.S. Application Data
(60) Provisional application No. 61/669,347, filed on Jul. 9, 2012.

(30) Foreign Application Priority Data
Jul. 6, 2012 (EP) 12175293.5

(51) Int. Cl. C12N 1/00; C12N 9/42

(52) U.S. Cl. CPC C12N 1/005; C12N 9/2437

(57) ABSTRACT

A method of inactivating the microbial host cell in a fermentation broth comprising an enzyme of interest and the microbial host cell producing the enzyme of interest comprising: a) Adding a fatty acid having a chain length of C4-C12 to the fermentation broth; and b) Mixing the fermentation broth for a sufficient period of time until the microbial host cell is inactivated.

### TECHNICAL FIELD

The present invention relates to a process of producing desired enzyme(s) as a crude product.

### BACKGROUND ART

Microbial host cells are today used extensively for producing enzymes by fermentation. Enzymes, especially for industrial use in the biofuel area, e.g., enzymes such as cellulases for converting plant material into syrups and/or fermentation products, are needed in large amounts. The enzymes can only be sold at relatively low prices. This renders the enzyme production cost an important factor for being successful in the market place.

One way of solving this problem is to produce a crude product, which means that the microbial host cells in the fermentation broth have been inactivated, but no recovery steps such as centrifugation and/or filtration have taken place.

### SUMMARY OF THE INVENTION

The inventors have found that the microbial host cells can be inactivated by using a fatty acid, so we claim:

A method of inactivating the microbial host cell in a fermentation broth comprising an enzyme of interest and the microbial host cell producing the enzyme of interest comprising
a) Adding a fatty acid having a chain length of C4-C12 to the fermentation broth; and
b) Mixing the fermentation broth for a sufficient period of time until the microbial host cell is inactivated.

The inventors have found that the microbial host cells can be inactivated by using a salt of a fatty acid, so we claim:

A method of inactivating the microbial host cell in a fermentation broth comprising an enzyme of interest and the microbial host cell producing the enzyme of interest comprising
a) Adding a salt of a fatty acid having a chain length of C4-C12 to the fermentation broth; and
b) Mixing the fermentation broth for a sufficient period of time until the microbial host cell is inactivated.

In a particular embodiment of the present invention the fatty acid has a chain length of C6-C8. In a more particular embodiment the fatty acid has a chain length of C8.

In a particular embodiment the salts of a fatty acid having a chain length of C6-C8 are preferred. In a more particular embodiment the salts of a fatty acid having a chain length of C8 are preferred.

### DETAILED DISCLOSURE OF THE INVENTION

It is the object of the present invention to provide a process of producing desired enzyme(s) as a crude product in industrial scale.

### Microbial Host Cells Capable of Producing the Enzyme(s) of Interest

The microbial host cell may be of any genus. The desired enzyme(s) may be homologous or heterologous to the host cell capable of producing the enzyme(s) of interest.

The term “homologous enzyme” means an enzyme encoded by a gene that is derived from the host cell in which it is produced.

The term “heterologous enzyme” means an enzyme encoded by a gene which is foreign to the host cell in which it is produced.

The term “recombinant host cell”, as used herein, means a host cell which harbours gene(s) encoding the desired enzyme(s) and is capable of expressing said gene(s) to produce the desired enzyme(s). The desired enzyme(s) coding gene(s) may be transformed, transfected, transducted, or the like, into the recombinant host cell using techniques well known in the art.

When the desired enzyme is a heterologous enzyme, the recombinant host cell capable of producing the desired enzyme is preferably of fungal or bacterial origin. The choice of recombinant host cell will to a large extent depend upon the gene coding for the desired enzyme and the source of said enzyme.

The term “wild-type host cell”, as used herein, refers to a host cell that natively harbours gene(s) coding for the desired enzyme(s) and is capable of expressing said gene(s).

A “mutant thereof” may be a wild-type host cell in which one or more genes have been deleted, e.g., in order to enrich the desired enzyme preparation.

A mutant wild-type host cell may also be a wild-type host cell transformed with one or more additional genes coding for additional enzymes in order to introduce one or more additional enzyme activities into the desired enzyme complex or preparation natively produced by the wild-type host cell.

The additional enzyme may be the same or another enzyme molecule.

The mutant wild-type host cell may also have additional homologous enzyme coding genes transformed, transfected, transducted, or the like, preferably integrated into the genome, in order to increase expression of that gene to produce more enzyme.

In a preferred embodiment, the recombinant or wild-type microbial host cell is a bacterium or a fungus.

The microbial host cell may be a yeast cell such as a Candida, Hansenula, Kluyveromyces, Pichia, Saccharomyces, Schizosaccharomyces or Yarrowia strain. In another aspect, the strain is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis strain.

The microbial host cell may be a filamentous fungal strain such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria strain.

In another aspect, the strain is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Schizosaccharomyces pombe, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride strain.

In one aspect, the fungal host cell is a strain selected from the group consisting of Candida, Hansenula, Kluyveromyces, Pichia, Saccharomyces, Schizosaccharomyces, Yarrowia, Acremonium, Aspergillus, Fusarium, Humicola, Mucor, Myceliophthora, Neurospora, Penicillium, Thielavia, Tolypocladium, and Trichoderma.

In a more preferred embodiment, the filamentous fungal host cell is selected from the group consisting of Trichoderma and Aspergillus host cells, in particular a strain of Trichoderma harzianum, Trichoderma koningii, Trichoderma longibrachiatum, Trichoderma reesei, Trichoderma viridel, Aspergillus awamori, Aspergillus fumigatus, Aspergillus foetidus, Aspergillus japonicus, Aspergillus kawachii, Aspergillus nidulans, Aspergillus niger, Aspergillus tubigensis or Aspergillus oryzae, especially a strain of Trichoderma reesei.

In another preferred embodiment, the recombinant or wild-type microbial host cell is a bacterium. Examples of microbial host cells include the ones selected from the group comprising gram positive bacteria such as a Bacillus, Clostridium, Enterococcus, Geobacillus, Lactobacillus, Lactococcus, Oceanobacillus, Staphylococcus, Streptococcus, or Streptomyces, or a Gram-negative bacteria such as a Campylobacter, Escherichia, Flavobacterium, Fusobacterium, Helicobacter, Ilyobacter, Neisseria, Pseudomonas, Salmonella, or Ureaplasma.

In one aspect, the bacterial host cell is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis.

In another aspect, the bacterial host cell is a Streptococcus equisimilis, Streptococcus pyogenes, Streptococcus uberis, or Streptococcus equi subspecies Zooepidemicus.

In another aspect, the bacterial host cell is a Streptomyces achromogenes, Streptomyces avermitilis, Streptomyces coelicolor, Streptomyces griseus, Steptomyces thermoviolaceus, Streptomyces fusca, Steptomyces harzianum or Streptomyces lividans strain.

In another aspect, the bacterial host cell is Escherichia coli.

In another aspect, the bacterial host cell is selected from the group consisting of Bacillus, Streptomyces, Escherichia and Pseudomonas.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

### Enzyme of Interest

The enzyme in the context of the present invention may be any enzyme or combination of different enzymes obtainable by fermentation. Accordingly, when reference is made to “an enzyme”, this will in general be understood to include both a single enzyme and a combination of more than one enzyme.

It is to be understood that enzyme variants (produced, for example, by recombinant techniques) are included within the meaning of the term “enzyme”.

Accordingly the types of enzymes which may appropriately be incorporated in the enzyme product of the invention include oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

Hydrolases of relevance for the present invention include the following (EC numbers in parentheses):

α-amylases (3.2.1.1), β-amylases (3.2.1.2), glucan 1,4-α-glucosidases (3.2.1.3), cellulases (3.2.1.4), endo-1,3(4)-β-glucanases (3.2.1.6), endo-1,4-β-xylanases (3.2.1.8), dextranases (3.2.1.11), chitinases (3.2.1.14), polygalacturonases (3.2.1.15), lysozymes (3.2.1.17), lipases (EC 3.1.1.3), phytases (EC 3.1.3.-), e.g. 3-phytases (EC 3.1.3.8) and 6-phytases (EC 3.1.3.26), β-glucosidases (3.2.1.21), α-galactosidases (3.2.1.22), β-galactosidases (3.2.1.23), amylo-1,6-glucosidases (3.2.1.33), xylan 1,4-β-xylosidases (3.2.1.37), glucan endo-1,3-β-D-glucosidases (3.2.1.39), α-dextrin endo-1,6-α-glucosidases (3.2.1.41), sucrose α-glucosidases (3.2.1.48), glucan endo-1,3-α-glucosidases (3.2.1.59), glucan 1,4-β-glucosidases (3.2.1.74), glucan endo-1,6-β-glucosidases (3.2.1.75), arabinan endo-1,5-α-L-arabinosidases (3.2.1.99), lactases (3.2.1.108), chitosanases (3.2.1.132) and xylose isomerases (5.3.1.5).

The enzyme(s) produced according to the invention may be any enzyme(s). Preferred enzymes are hydrolases including especially cellulases, hemicellulases, amylases, glucoamylases, xylanases, beta-xylosidases, beta-glucosidases, phytases, lipases or any other hydrolases, especially enzymes used for converting plant materials into syrups and fermentation substrates, e.g., converted by a yeast into ethanol.

According to the present invention, an enzyme selected from the group consisting of cellulases, xylanases, beta-xylosidases and beta-glucosidases is particularly preferred.

In one embodiment, the enzyme of interest is a mono-component enzyme. In another embodiment, the enzymes of interest are an enzyme preparation or enzyme complex consisting of two of more enzymes derived from a wild-type host cell or a mutant thereof.

An example of an enzyme complex is the well known Trichoderme reesei cellulase complex comprising endoglucanase, xylanase, exo-cellobiohydrolase and beta-glucosidase. An example of an enzyme preparation is the above mentioned cellulase complex where one or more enzyme encoding genes, e.g., endoglucanase gene(s), have been deleted from the wild-type host cell. A cellulase complex or preparation may be produced by a wild-type host cell or mutant thereof. In one embodiment the enzyme is produced recombinantly in a suitable recombinant host cell different from the donor cell from which the enzyme coding gene is derived. The desired enzyme may be extracellular or intracellular. Extracellular enzymes are preferred. A desired enzyme may also be a variant of a wild-type enzyme.

### Cellulase and Hemicellulase

A cellulase and/or a hemicellulase complex may be the desired enzyme produced according to the invention.

Hemicellulases include xylanases, arabinofuranosidases, acetyl xylan esterases, glucuronidases, endo-galactanase, mannases, endo- or exo-arabinases, and exo-galactanses.

Cellulases include those of bacterial or fungal origin. Chemically modified or protein engineered variants are included. Suitable cellulases include cellulases from the genera Bacillus, Penicillium, Thermonospore, Clostridium, Cellulomonas, Hypocrea, Pseudomonas, Humicola, Fusarium, Thielavia, Acremonium, and Trichoderma, e.g., fungal cellulases produced by Humicola insolens, Myceliophthora thermophila, Thielavia terrestris, Fusarium oxysporum, and Trichoderma reesei.

In a preferred embodiment, the desired enzyme is the cellulase complex which is homologously produced by Trichoderma reesei.

In another preferred embodiment, the desired enzyme is a cellulase and hemicellulase complex produced heterologously in Trichoderma reesei, wherein one or more hydrolases foreign to Trichoderma reesei are produced, e.g., Cellic® CTec products produced by Novozymes A/S.

In another embodiment, the desired enzyme is the cellulase complex which is homologously produced by Humicola insolens.

### Amylase

An amylase may be the desired enzyme produced according to the invention. Amylases include alpha-amylases, beta-amylases and maltogenic amylases.

An alpha-amylase may be derived from the genus Bacillus, such as, derived from a strain of B. licheniformis, B. amyloliquefaciens, B. sultilis and B. stearothermophilus. Other alpha-amylases include alpha-amylase derived from the strain Bacillus sp. NCIB 12289, NCIB 12512, NCIB 12513 or DSM 9375, all of which are described in detail in WO 95/26397, or the alpha-amylase described by Tsukamoto et al., Biochemical and Biophysical Research Communications, 151 (1988), pp. 25-31.

Other alpha-amylases include alpha-amylases derived from a filamentous fungus, preferably a strain of Aspergillus, such as, Aspergillus oryzae and Aspergillus niger.

In a preferred embodiment, the desired enzyme is an alpha-amylase derived from Aspergillus oryzae such as the one having the amino acid sequence shown in SEQ ID NO: 10 in WO 96/23874.

The desired enzyme may also be an alpha-amylase derived from A. niger, especially the one disclosed as “AMYA_ASPNG” in the Swiss-prot/TeEMBL database under the primary accession no. P56271.

The desired enzyme may also be a beta-amylase, such as any of plants and micro-organism beta-amylases disclosed in W. M. Fogarty and C. T. Kelly, Progress in Industrial Microbiology, vol. 15, pp. 112-115, 1979.

The desired enzyme may also be a maltogenic amylase. A “maltogenic amylase” (glucan 1,4-alpha-maltohydrolase, E.C. 3.2.1.133) is able to hydrolyze amylose and amylopectin to maltose in the alpha-configuration. A maltogenic amylase of interest is the one derived from Bacillus stearothermophilus strain NCIB 11837. Maltogenic alpha-amylases are described in U.S. Pat. Nos. 4,598,048; 4,604,355; and 6,162,628.

### Glucoamylase

A glucoamylase may be the enzyme of interest produced according to the invention. A glucoamylase may be derived from any suitable source, e.g., derived from a micro-organism or a plant. Preferred glucoamylases are of fungal or bacterial origin, e.g., selected from the group consisting of Aspergillus glucoamylases, in particular the A. niger G1 or G2 glucoamylases (Boel et al., 1984, EMBO J. 3:5, p. 1097-1102); the A. awamori glucoamylase (WO 84/02921), A. oryzae glucoamylase (Agric. Biol. Chem., 1991, 55:4, p. 941-949). Other glucoamylases include Athelia rolfsii (previously denoted Corticium rolfsii) glucoamylase (see U.S. Pat. No. 4,727,026 and (Nagasaka, Y. et al. (1998) Purification and properties of the raw-starch-degrading glucoamylases from Corticium rolfsii, Appl Microbiol Biotechnol 50:323-330), Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases include glucoamylases from the genus Clostridium, in particular C. thermoamylolyticum (EP 135,138), and C. thermohydrosulfuricum (WO 86/01831).

### Fermentation Broth

The term “fermentation broth” as used in the context of the present invention is to be understood as an aqueous composition, comprising both an enzyme of interest and the production strain which during a fermentation process has produced the enzyme of interest.

The composition of the fermentation broth is complex consisting of anything that ends up in the fermentation broth. This includes:

1 Raw substrates

2 Fermentation products

3 Microorganisms and derivative components

4 Chemical additives added to the fermentor

5 Gases such as oxygen and other metabolic gases

The present invention may be useful for any submerged fermentation in industrial scale, e.g. for any fermentation having a culture media of at least 10,000 liters, preferably of at least 20,000 liters, more preferably of at least 50,000 liters, more preferably of at least 100,000 liters, even more preferably of at least 200,000 liters, in particular with a culture media of from 20,000 liters to 2,000,000 liters; especially with a culture media of from 50,000 liters to 500,000 liters.

The host cell may be fermented by any method known in the art. The fermentation medium may be a complex medium comprising complex nitrogen and/or carbon sources, such as soybean meal, soy protein, soy protein hydrolysate, cotton seed meal, corn steep liquor, yeast extract, casein, casein hydrolysate, potato protein, potato protein hydrolysate, molasses, and the like. The fermentation medium may be a chemically defined media, e.g., as defined in WO 98/37179.

The fermentation may be performed as a batch, a fed-batch, a repeated fed-batch or a continuous fermentation process; in particular as a fed-batch fermentation process.

### Fatty Acids

A fatty acid is a carboxylic acid with an aliphatic tail (chain), which is either saturated or unsaturated. Fatty acids that have double bonds are known as unsaturated. Fatty acids without double bonds are known as saturated. They differ in length as well. Fatty acids are usually derived from triglycerides or phospholipids. When they are not attached to other molecules, they are also known as “free” fatty acids.

### Length of Free Fatty Acid Chains:

Fatty acid chains differ by length, often categorized as short, medium, or long.
Short-chain fatty acids (SOFA) are fatty acids with aliphatic tails of fewer than six carbons.
Medium-chain fatty acids (MCFA) are fatty acids with aliphatic tails of 6-12 carbons.
Long-chain fatty acids (LCFA) are fatty acids with aliphatic tails longer than 12 carbons.

Below is a list of saturated fatty acids:

| Common Name | Systematic Name | Structural Formula | Chain length |
|---|---|---|---|
| Propionic acid | Propanoic acid | CH₃CH₂COOH | C3 |
| Butyric acid | Butanoic acid | CH₃(CH₂)₂COOH | C4 |
| Valeric acid | Pentanoic acid | CH₃(CH₂)₃COOH | C5 |
| Caproic acid | Hexanoic acid | CH₃(CH₂)₄COOH | C6 |
| Enanthic acid | Heptanoic acid | CH₃(CH₂)₅)COOH | C7 |
| Caprylic acid | Octanoic acid | CH₃(CH₂)₆COOH | C8 |
| Pelargonic acid | Nonanoic acid | CH₃(CH₂)₇COOH | C9 |
| Capric acid | Decanoic acid | CH₃(CH₂)₈COOH | C10 |
| Undecylic acid | Undecanoic acid | CH₃(CH₂)₉COOH | C11 |
| Lauric acid | Dodecanoic acid | CH₃(CH₂)₁₀COOH | C12 |
| Tridecylic acid | Tridecanoic acid | CH₃(CH₂)₁₁COOH | C13 |
| Myristic acid | Tetradecanoic acid | CH₃(CH₂)₁₂COOH | C14 |
| Pentadecylic acid | Pentadecanoic acid | CH₃(CH₂)₁₃COOH | C15 |
| Palmitic acid | Hexadecanoic acid | CH₃(CH₂)₁₄COOH | C16 |
| Margaric acid | Heptadecanoic acid | CH₃(CH₂)₁₅COOH | C17 |
| Stearic acid | Octadecanoic acid | CH₃(CH₂)₁₆COOH | C18 |
| Nonadecylic acid | Nonadecanoic acid | CH₃(CH₂)₁₇COOH | C19 |
| Arachidic acid | Eicosanoic acid | CH₃(CH₂)₁₈COOH | C20 |
| Heneicosylic acid | Heneicosanoic acid | CH₃(CH₂)₁₉COOH | C21 |
| Behenic acid | Docosanoic acid | CH₃(CH₂)₂₀COOH | C22 |
| Tricosylic acid | Tricosanoic acid | CH₃(CH₂)₂₁COOH | C23 |
| Lignoceric acid | Tetracosanoic acid | CH₃(CH₂)₂₂COOH | C24 |
| Pentacosylic acid | Pentacosanoic acid | CH₃(CH₂)₂₃COOH | C25 |
| Cerotic acid | Hexacosanoic acid | CH₃(CH₂)₂₄COOH | C26 |
| Heptacosylic acid | Heptacosanoic acid | CH₃(CH₂)₂₅COOH | C27 |
| Montanic acid | Octacosanoic acid | CH₃(CH₂)₂₆COOH | C28 |
| Nonacosylic acid | Nonacosanoic acid | CH₃(CH₂)₂₇COOH | C29 |
| Melissic acid | Triacontanoic acid | CH₃(CH₂)₂₈COOH | C30 |
| Henatriacontylic acid | Henatriacontanoic acid | CH₃(CH₂)₂₉COOH | C31 |
| Lacceroic acid | Dotriacontanoic acid | CH₃(CH₂)₃₀COOH | C32 |
| Psyllic acid | Tritriacontanoic acid | CH₃(CH₂)₃₁COOH | C33 |
| Geddic acid | Tetratriacontanoic acid | CH₃(CH₂)₃₂COOH | C34 |
| Ceroplastic acid | Pentatriacontanoic acid | CH₃(CH₂)₃₃COOH | C35 |
| Hexatriacontylic acid | Hexatriacontanoic acid | CH₃(CH₂)₃₄COOH | C36 |

According to the present invention, any liquid fatty acid is preferred.

According to the present invention, the fatty acids with an aliphatic tail of C3 to C11 (the fatty acid has a chain length of C4-C12) are preferred:

| | | | |
|---|---|---|---|
| Butyric acid | Butanoic acid | CH₃(CH₂)₂COOH | C4 |
| Valeric acid | Pentanoic acid | CH₃(CH₂)₃COOH | C5 |
| Caproic acid | Hexanoic acid | CH₃(CH₂)₄COOH | C6 |
| Enanthic acid | Heptanoic acid | CH₃(CH₂)₅)COOH | C7 |
| Caprylic acid | Octanoic acid | CH₃(CH₂)₆COOH | C8 |
| Pelargonic acid | Nonanoic acid | CH₃(CH₂)₇COOH | C9 |
| Capric acid | Decanoic acid | CH₃(CH₂)₈COOH | C10 |
| Undecylic acid | Undecanoic acid | CH₃(CH₂)₉COOH | C11 |
| Lauric acid | Dodecanoicacid | CH₃(CH₂)₁₀COOH | C12 |

In a particular embodiment the salts of C4-C12 are preferred.

According to the present invention, the fatty acids with an aliphatic tail of C4 to C7 (the fatty acid has a chain length of C5-C8) are especially preferred:

| | | | |
|---|---|---|---|
| Valeric acid | Pentanoic acid | CH₃(CH₂)₃COOH | C5 |
| Caproic acid | Hexanoic acid | CH₃(CH₂)₄COOH | C6 |
| Enanthic acid | Heptanoic acid | CH₃(CH₂)₅)COOH | C7 |
| Caprylic acic | Octanoic acid | CH₃(CH₂)₆COOH | C8 |

In a particular embodiment the salts of a fatty acid having a chain length of C5-C8 are preferred.

In a particular embodiment of the present invention the fatty acid has a chain length of C6-C8. In another particular embodiment the fatty acid has a chain length of C7-C8. In a more particular embodiment the fatty acid has a chain length of C8.

| | | | |
|---|---|---|---|
| Caproic acid | Hexanoic acid | CH₃(CH₂)₄COOH | C6 |
| Enanthic acid | Heptanoic acid | CH₃(CH₂)₅)COOH | C7 |
| Caprylic acid | Octanoic acid | CH₃(CH₂)₆COOH | C8 |

In a particular embodiment the salts of a fatty acid having a chain length of C6-C7 are preferred. In a more particular embodiment the salts of a fatty acid having a chain length of C8 are preferred.

In a particular embodiment the salts of a fatty acid having a chain length of C6-C8 are preferred. In a more particular embodiment the salts of a fatty acid having a chain length of C8 are preferred.

According to the present invention, especially hexanoic acid or octanoic acid is preferred. In a particular embodiment the salts of hexanoic acid and/or octanoic acid are preferred. Hexanoic acid has a melting point of—3.4 degrees Celsius, and octanoic acid has a melting point of 16.7 degrees Celsius.

### Inactivation

Fatty acids have a strong germicidal effect at low concentrations and are very effective against bacteria and yeast and moulds. The fatty acid will inactivate and/or reduce the living organisms present in the fermentation broth.

The fatty acid may be added in an amount of 0.01% to 5.0% (w/w) per kg fermentation broth; in particular 0.01% to 4.0% (w/w) per kg fermentation broth; in particular 0.01% to 3.0% (w/w) per kg fermentation broth; in particular 0.01% to 2.0% (w/w) per kg fermentation broth; in particular 0.01% to 1.0% (w/w) per kg fermentation broth; in particular 0.02% to 1.0% (w/w) per kg fermentation broth; in particular 0.03% to 1.0% (w/w) per kg fermentation broth; in particular 0.04% to 1.0% (w/w) per kg fermentation broth; in particular 0.05% to 1.0% (w/w) per kg fermentation broth.

After the fatty acid has been added, the pH may be adjusted. In a preferred embodiment, the pH is adjusted to a pH in the range of pH 3.0 to pH 7.0; in particular the pH is adjusted to a pH in the range of pH 3.0 to pH 6.5; in particular the pH is adjusted to a pH in the range of pH 3.0 to pH 6.0; in particular the pH is adjusted to a pH in the range of pH 3.0 to pH 5.5; in particular the pH is adjusted to a pH in the range of pH 3.0 to pH 5.0; in particular the pH is adjusted to a pH in the range of pH 3.5 to pH 5.0; in particular the pH is adjusted to a pH in the range of pH 4.0 to pH 5.0; especially the pH is adjusted to a pH around 4.5.

The pH may be adjusted by using any acid or base known in the art, e.g., acetic acid or sodium hydroxide.

The fatty acid is mixed with the fermentation broth for a sufficient period of time. Samples may be taken out at various times in order to find the needed hours in order to inactive the microbial host cell.

The fermentation broth with the fatty acid is mixed for a time period of up to 40 hours; e.g. for a time period of up to 1 min.; e.g. for a time period of up to 2 min.; e.g. for a time period of up to 3 min.; e.g. for a time period of up to 4 min.; e.g. for a time period of up to 5 min.; e.g. for a time period of up to 6 min.; e.g. for a time period of up to 7 min.; e.g. for a time period of up to 8 min.; e.g. for a time period of up to 9 min.; e.g. for a time period of up to 10 min.; e.g. for a time period of up to 11 min.; e.g. for a time period of up to 12 min.; e.g. for a time period of up to 13 min.; e.g. for a time period of up to 14 min.; e.g. for a time period of up to 15 min.; e.g. for a time period of up to 16 min.; e.g. for a time period of up to 17 min.; e.g. for a time period of up to 18 min.; e.g. for a time period of up to 19 min.; e.g. for a time period of up to 20 min.; e.g. for a time period of up to 21 min.; e.g. for a time period of up to 22 min.; e.g. for a time period of up to 23 min.; e.g. for a time period of up to 24 min.; e.g. for a time period of up to 25 min.; e.g. for a time period of up to 26 min.; e.g. for a time period of up to 27 min.; e.g. for a time period of up to 28 min.; e.g. for a time period of up to 29 min.; in particular for a time period of 0.5-40 hours.

The temperature will typically be room temperature. The mixing may be done as known in the art, e.g., by stirring. The mixing should be done in such a way that the entire fermentation broth is being circulated and well mixed.

### Applications

The method according to the present invention may be used in many industrial applications where a crude enzyme solution may be adequate, e.g., in Bio Ethanol applications (e.g. Biomass conversion).

### EXAMPLES

### Inactivation of the Microbial Host Cells

The fatty acid is added to the fermentation broth at various concentrations (0.09% (w/w); 0.28% (w/w); 0.46% (w/w); 0.65% (w/w)).

pH is adjusted to 4.5 using an aqueous solution of acetic acid (CAS 64-19-7) and/or aqueous sodium hydroxide (CAS 1310-73-2).

The fermentation broth with the various concentrations of the fatty acid is left at pH 4.5 for 24 hrs with constant stirring at room temperature.

The fermentation broth is tested after 24 hrs and inactivation of the microbial host cells is successful if there is no growth on agar plates—samples are incubated for 4 days at 30 degrees Celsius.

The crude enzyme product is ready for use.

### Example 1

Production of Cellulase Enzymes in Trichoderma reesei Followed by Inactivation of the Production Strain

The fermentations are run using Trichoderma reesei as the microbial host cell. Trichoderma reesei strains producing cellulases are publicly available, e.g., from DSMZ.

Glycerol freezer stocks are used as inoculum for the seed flasks. Seed flasks are grown as shown in the table below. 10% of the main tank volume is used (app. 10,000 liters) in the seed process.

Trace Metals Preparation

| Components | g/L |
|---|---|
| FeCl₃•6H₂0 | 216 |
| ZnSO₄•7H₂O | 58 |
| MnSO₄•H₂O | 27 |
| CuSO₄•5H₂O | 10 |

Seed Substrate Mix

| Components | g/kg |
|---|---|
| Glucose syrup (73% w/w) | 27 |
| Corn Steep Liqour | 19 |
| (NH₄)₂SO₄ | 1.5 |
| KH₂PO₄ | 2.1 |
| CaCO₃ | 0.2 |
| MgSO₄•7H₂O | 0.4 |
| Citric acid | 0.05 |
| Trace Metals | 0.06 |
| Antifoam oil | 0.4 |

Sterilisation process: Adjust pH to 5.0 with 25% NaOH or 25% H3PO4. Raise temperature to 123 degrees C. for 1.5 h.

Post sterilisation: Adjust temperature to 28 degrees C. Adjust pH to 5.0 with 25% NaOH or 25% H3PO4.

Inoculation: Inoculate with spores of Trichoderma reesei.

### Fermentation Phase:

Temperature: 28 degrees C.

Pressure: 1 bar over atmospheric pressure

Agitation: 100 rpm

Air: 15000 Nliters/min

Fermentation is complete when pH falls below 4.5 (after approximately 40 hours).

Main Tank:

Main Tank Substrate Mix

| (final concentrations) | g/kg |
|---|---|
| (NH₄)₂SO₄ | 3.7 |
| CaCO₃ | 0.8 |
| K₂SO₄ | 0.9 |
| Na₂SO₄ | 0.3 |
| MgSO₄•7H₂O | 0.9 |
| Citric acid | 0.27 |
| Trace Metals | 0.16 |
| Antifoam oil | 0.25 |

Inoculation: The seed material produced as described above is pumped into the main tank.

### Feed System:

A feed system with carbohydrate compound(s) like the feed system disclosed in WO 2006/125068 is used.

The feed is prepared and stored in a standard stirred tank with a sterilization of 123 degrees C. for 1.5 h. The feed is added gradually. The fermentation is complete when the target product concentration is achieved.

### Inactivation of the Microbial Host Cells

Hexanoic acid (100% sol., CAS 142-62-1) was added to the fermentation broth at various concentrations (0.09% (w/w); 0.28% (w/w); 0.46% (w/w); 0.65% (w/w)).

pH was adjusted to 4.5 using an aqueous solution of acetic acid (CAS 64-19-7) and/or aqueous sodium hydroxide (CAS 1310-73-2).

The fermentation broth with the various concentrations of hexanoic acid was left at pH 4.5 for 24 hrs with constant stirring at room temperature.

After 24 hrs the Trichoderma reesei microbial host cells were inactivated (no growth on agar plates—samples were incubated for 4 days at 30 degrees Celsius).

The activity of the enzyme of interest (cellulase product) was not significantly reduced by the fatty acid treatment.

The crude enzyme product is ready for use.

1. A method of inactivating the microbial host cell in a fermentation broth comprising an enzyme of interest and the microbial host cell producing the enzyme of interest comprising
a) Adding a fatty acid having a chain length of C4-C12 to the fermentation broth; and
b) Mixing the fermentation broth for a sufficient period of time until the microbial host cell is inactivated.

2. The method of claim 1, wherein the fatty acid has a chain length of C6-C12.

3. The method of claim 1, wherein the fatty acid has a chain length of C6-C8.

4. The method of claim 1, wherein the fatty acid has a chain length of C7-C8.

5. The method of claim 1, wherein the fatty acid has a chain length of C8.

6. The method according to claim 1, wherein the microbial host cell is a bacterium or a fungus.

7. The method according to claim 6, wherein the bacterium is a strain selected from the group consisting of Bacillus, Streptomyces, Escherichia and Pseudomonas.

8. The method according to claim 6, wherein the fungus is a strain selected from the group consisting of Candida, Hansenula, Kluyveromyces, Pichia, Saccharomyces, Schizosaccharomyces, Yarrowia, Acremonium, Aspergillus, Fusarium, Humicola, Mucor, Myceliophthora, Neurospora, Penicillium, Thielavia, Tolypocladium, and Trichoderma.

9. The method according to claim 1, wherein the enzyme is selected from the group consisting of oxidoreductases, transferases, hydrolases, lyases, isomerases and ligases.

10. The method according to claim 1, wherein the enzyme is selected from the group consisting of cellulases, xylanases, beta-xylosidases and beta-glucosidases.

11. The method according to claim 1, wherein the fatty acid is a liquid fatty acid.

12. The method according to claim 1, wherein the fatty acid is hexanoic acid or octanoic acid.

13. The method according to claim 1, wherein the fatty acid is added in an amount of 0.01%-5.0% (w/w) per kg fermentation broth.

14. The method according to claim 1, wherein the mixing lasts up to 40 hours.

15. The method according to claim 1, wherein the mixing lasts from 0.5-40 hours.

16. A method of inactivating the microbial host cell in a fermentation broth comprising an enzyme of interest and the microbial host cell producing the enzyme of interest comprising
a) Adding a salt of a fatty acid having a chain length of C4-C12 to the fermentation broth; and
Mixing the fermentation broth for a sufficient period of time until the microbial host cell is inactivated.
